(19) United States
(12) Patent Application Publication
(10) Pub. No.: US 2013/0237893 A1
(43) Pub. Date: Sep. 12, 2013

(54) Infant and Child Upper Extremity And Thoracic Stability Brace

(71) Applicant: Shzabette Elizabeth Knox, Platteville, WI (US)

(72) Inventor: Shzabette Elizabeth Knox, Platteville, WI (US)

(21) Appl. No.: 13/693,293

(22) Filed: Dec. 4, 2012

Related U.S. Application Data
(60) Provisional application No. 61/608,188, filed on Mar. 8, 2012.

(51) Int. Cl. A61F 5/01

(52) U.S. Cl. CPC A61F 5/0118
USPC 602/20

(57) ABSTRACT

A body recovery apparatus comprises a splint, a chest band, and an abdomen wrap band. The splint comprises a humerus support section, an ulna radius support section, and a flex section flexibly connecting the humerus support section to the ulna radius support section. The chest band comprises a first chest band fastener configured to fasten to at least one of an apparel of the user and a second chest band fastener of the chest band. The abdomen wrap band comprises an abdomen band comprising a first abdomen band fastener configured to fasten to at least one of the apparel of the user and a second abdomen band fastener of the abdomen band, and an ulna radius support section band connected to the abdomen band.

### REFERENCE TO RELATED APPLICATION

This application claims priority to U.S. Provisional Application No. 61/608,188, filed Mar. 8, 2012, entitled “Infant/child. Upper Extremity and Thoracic Stability Brace.”

### BACKGROUND OF THE INVENTION

The present invention relates generally to infant stabilizing braces and more specifically it relates to an infant and Child Upper Extremity and Thoracic Stability Brace for stabilizing a fractured or sprained wound in the thoracic region and/or upper extremity region of an infant/child.

### SUMMARY OF THE INVENTION

The invention generally relates to an infant stabilizing brace of which the main elements are Olivia's Splint, which shall mean: a splint that incorporates mechanical statics, restriction of a body, and a positioning tool to provide increased protection, release of pressure from the joint, room for anticipated growth and guard against negative inflictions; the arm and chest band; and the tummy wrap. The invention uses Olivia's Hybrid Model. Olivia's Hybrid Model shall mean: an application that creates an advantageous symbiotic relationship that incorporates the principles of a cast, splint, and brace, while working against negatively perceived inflictions and simultaneously combining the objectives of mechanical statics, positioning, pressure, and anticipated expansion of the subject's physical anatomy for a specific purpose.

There has thus been outlined, rather broadly, some of the features of the invention in order that the detailed description thereof may be better understood, and in order that the present contribution to the art may be better appreciated. There are additional features of the invention that will be described hereinafter.

In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

Another object is to provide an infant and Child Upper Extremity and Thoracic Stability Brace that targets dystocia conditions and other child and/or infant injuries, conditions, and disorders in the thoracic and upper extremities regions of infants.

An object is to provide an infant and Child Upper Extremity and Thoracic Stability Brace for stabilizing a fractured or sprained wound in the thoracic region and/or upper extremity region of an infant or child.

Another object is to provide an infant and Child Upper Extremity and Thoracic Stability Brace that is designed to conveniently locate the brachial artery for measuring and/or monitoring purposes without interfering with the natural pulse rhythms of the user.

Another object is to provide an infant and Child Upper Extremity and Thoracic Stability Brace that provides an aligning splint for a fractured or broken bone.

Another object is to provide an infant and Child Upper Extremity and Thoracic Stability Brace that provides a support for muscular or ligament trauma.

Another object is to provide an infant and Child Upper Extremity and Thoracic Stability Brace that is safe to use.

Another object is to provide an infant and Child Upper Extremity and Thoracic Stability Brace that is comfortable to wear without affecting the purpose of the brace.

Another object is to provide an infant and Child Upper Extremity and Thoracic Stability Brace that is designed to decrease recovery time.

Another object is to provide an infant and Child Upper Extremity and Thoracic Stability Brace that is easy and convenient to use without affecting the purpose brace.

Another object is to provide an infant and Child Upper Extremity and Thoracic Stability Brace that is easy and convenient to use when handling the infant/child.

Another object is to provide an Infant and Child Upper Extremity and Thoracic Stability Brace that incorporates mechanical statics.

Another object is to provide an infant and Child Upper Extremity and Thoracic Stability Brace that incorporates restriction of a body.

Another object is to provide an infant and Child Upper Extremity and Thoracic Stability Brace that incorporates a positioning tool to provide increased protection.

Another object is to provide an Infant and Child Upper Extremity and Thoracic Stability Brace that incorporates release of pressure from the joint.

Another object is to provide an Infant and Child Upper Extremity and Thoracic Stability Brace that incorporates room for anticipated growth.

Another object is to provide an infant and Child Upper Extremity and Thoracic Stability Brace that uses Olivia's Hybrid Model.

Another object is to provide an infant and Child Upper Extremity and Thoracic Stability Brace that mimics nursing and cradling positions.

Another object is to provide an infant and Child Upper Extremity and Thoracic Stability Brace that minimizes further damage to the wound or fracture.

Another object is to provide an infant and Child Upper Extremity and Thoracic Stability Brace that guard against negative inflictions.

Another object is to provide an infant and Child Upper Extremity and Thoracic Stability Brace that is designed to prevent irritants from reaching the skin.

Another object is to provide an infant and Child Upper Extremity and Thoracic Stability Brace that minimizes pain felt by the user.

Another object is to provide an infant and Child Upper Extremity and Thoracic Stability Brace that is simple and easy to use between Practitioners, parents, and guardians, without compromising the purpose of the brace.

According to one aspect of the present invention, a body recovery apparatus comprises: a splint comprising a humerus support section having a curved cross section, an ulna radius support section having a curved cross section, and a flex section flexibly connecting the humerus support section to the ulna radius support section, wherein the humerus support section, ulna radius support section, and flex section are shaped and configured to define a decompression hole configured to align with a user's olecranon when the splint is applied to an arm of the user; a chest band configured to enwrap the humerus support section of the splint and comprising a first chest band fastener configured to fasten to at least one of an apparel of the user and a second chest band fastener of the chest band; and an abdomen wrap band comprising an abdomen band comprising a first abdomen band fastener configured to fasten to at least one of the apparel of the user and a second abdomen band fastener of the abdomen band, and an ulna radius support section band connected to the abdomen band and comprising an ulna radius support section fastener configured to fasten to the ulna radius support section.

In one aspect, at least one of the fasteners comprises a hook-and-loop fastener. In one aspect, the chest band comprises the second chest band fastener and the first chest band fastener is configured to fasten to the second chest band fastener. In one aspect, the first chest band fastener is configured to fasten to the apparel of the user. In one aspect, the abdomen band comprises the second abdomen band fastener and the first abdomen band fastener is configured to fasten to the second abdomen band fastener. In one aspect, the abdomen torso band fastener is configured to fasten to the apparel of the user. In one aspect, the apparatus may further comprise an absorbent material connected to at least one of the humerus support section and the ulna radius support section.

According to one aspect of the present invention, a method of healing a body comprises: providing the body recovery apparatus as described; applying the splint to the arm of the user; bending the splint at the flex section so that the humerus support section and ulna radius support section are angled relative to each other at an angle of between approximately 60 and 100 degrees; fastening the chest band around the humerus support section; fastening the first chest band fastener to at least one of the apparel of the user and the second chest band fastener of the chest band; fastening the first abdomen band fastener to at least one of the apparel of the user and the second torso band fastener of the torso band; and fastening the ulna radius support section fastener to the ulna radius support section.

In one aspect, the user is an infant having dystocia condition in at least one of a thoracic and upper extremity region. The method may further comprise completely encasing at least one of a circumference of the ulna region and a circumference of the humerus region. In one aspect, the four steps of fastening result in substantial immovability of the arm of the user, in one aspect, the step of fastening the first abdomen band fastener comprises fastening the first abdomen band fastener to the second abdomen band fastener at a back of the user. The method may further comprise accessing the brachial artery while the body healing apparatus is applied to the arm of the user. The method may further comprise adjusting the splint to achieve comfort of the user. The method may further comprise applying a scientific discipline of mechanical statics in executing the claimed steps.

Other objects and advantages of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is a perspective view of the invention shown in use. FIG. 1 illustrates a typical fully assembled infant and Child Upper Extremity and Thoracic Stability Brace applied to the infant/child. The brace includes sub-assembly sections that make up the entirety of the brace. FIG. 1 shows the illustrated assembly and how it will look on an infant or child. The Infant/Child Upper Extremity and Thoracic Stability Brace consist of Olivia's Splint item 12, the tummy wrap item 24 and the arm and chest band item 46.

FIG. 2 is a perspective view of Olivia's Splint which contains sub-elements ulna and radius support section item 16 and humerus support section item 22.

FIG. 3 is a perspective view of Olivia's Splint which contains sub-elements ulna and radius support section item 16 and the humerus support section item 22 illustrated in exemplary angled configuration.

FIG. 4 is a perspective view of Olivia's Splint which contains sub-elements ulna and radius support section item 16 and the humerus support section item 22 illustrated in a frontal view in an exemplary angled configuration. FIG. 4 is illustrated for better understanding of FIG. 2 and FIG. 3.

FIG. 5 is a top view of the invention item 24 tummy wrap only.

FIG. 6 is a perspective view of the invention item 24 tummy wrap only.

FIG. 7 is a top view of the invention item 46 arm and chest band only.

FIG. 8 is a perspective view of the invention item 46 arm and chest band only.

FIG. 9 is an alternative embodiment of the present invention. FIG. 9 illustrates a typical fully assembled Infant and Child Upper Extremity and Thoracic Stability Brace in the alternative embodiment view. The brace includes sub-assembly sections that make up the entirety of the brace. FIG. 9 also shows the illustrated assembly and how it will look on an infant or child. The Infant/Child Upper Extremity and Thoracic Stability Brace consists of Olivia's Splint item 12, the tummy wrap item 24 and the arm and chest band item 46.

### DETAILED DESCRIPTION OF THE INVENTION

### A. Overview

Turning now descriptively to the drawings, in which similar reference characters denote similar elements throughout the several views, the figures illustrate the main elements of my invention which are Olivia's Splint; the arm and chest band; and the tummy wrap.

### B. Olivia's Splint

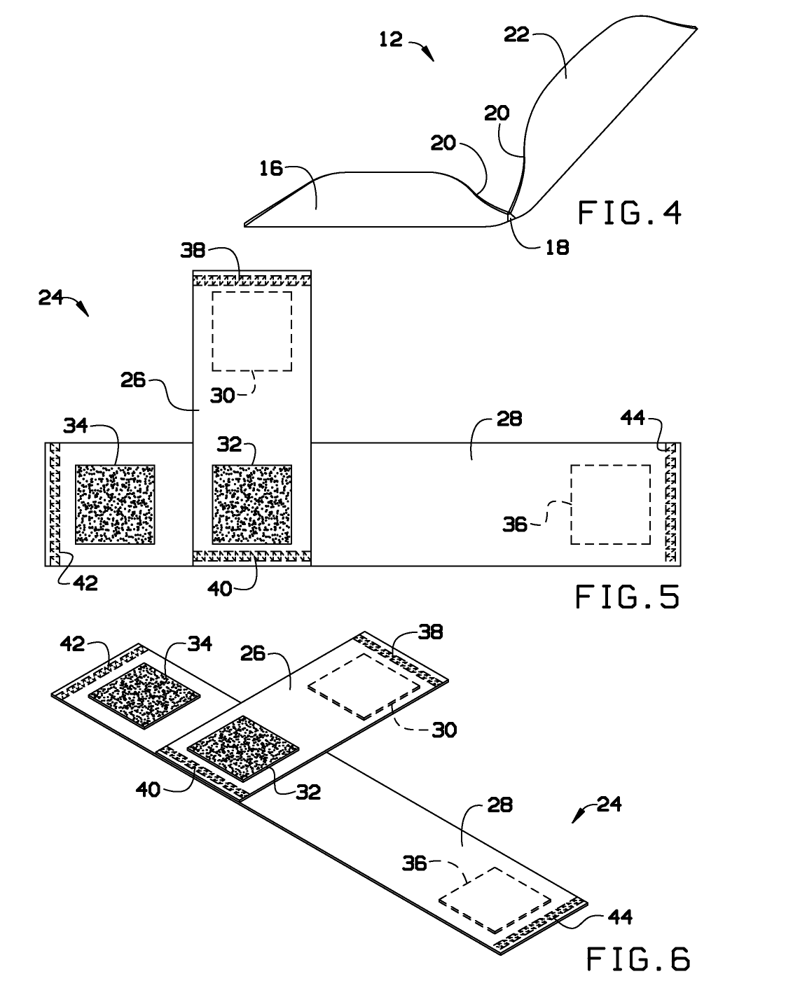

In the preferred central stabilizing brace, conventional orthopedic material(s) is pressed, cut and/or fabricated to conform to the profile of the user's extremity. The conventional orthopedic material(s) are measured to contour to the profile of a user to establish Olivia's splint item 12 and for better understanding shown in illustrations of FIG. 3, and FIG. 4. The dimensions of Olivia's Splint are roughly six and three quarter inches long, the width of the splint, however has two different dimensions. The ulna and radius support section of the splint item 16 shown in FIG. 2 is roughly one and six eighths inches wide. The humerus support section of the splint item 22 shown in FIG. 2 is roughly three and one eighth inches wide. The ulna and radius support section item 16 and humerus support section item 22 portion of the splint is concaved but does not limit the scope or the functionality of the invention. The manufacturing process of Olivia's Splint item 12 will use methods commonly utilized in orthopedic device(s) and material(s). Olivia's Splint item 12 will force the user's arm to bend at roughly a ninety degree angle holding the bones, and/or muscular ligaments in their rightful settings shown in illustration FIG. 3 and for better understanding FIG. 4. Olivia's Splint item 12 will keep the user's arm stable to prevent the user from causing further damage to the injury. Olivia's Splint item 12 will also prevent misalignment of the humerus, radius, and ulna bones. Olivia's Splint item 12 will stabilize and support the healing of fractures, contusions, or wounds that occurred on/in the user's arm or wrist. Olivia's Splint item 12 was created in mind to provide a safe orthopedic brace for infants and/or children and made of non-hazardous material for infants and/or children.

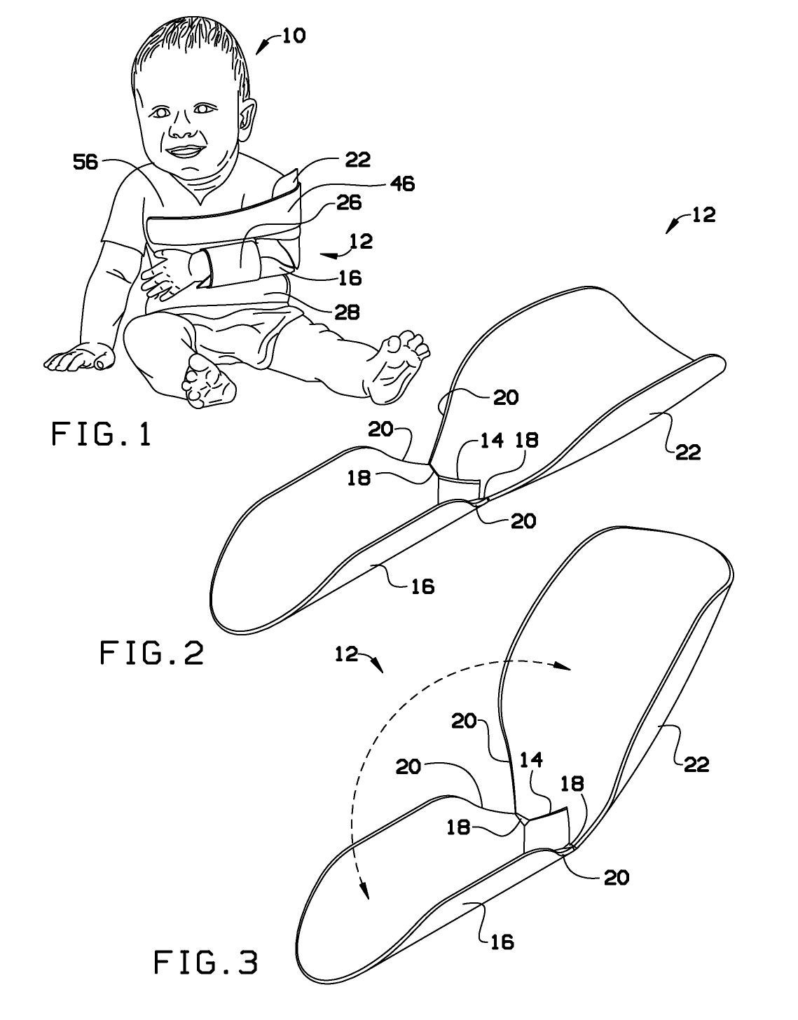

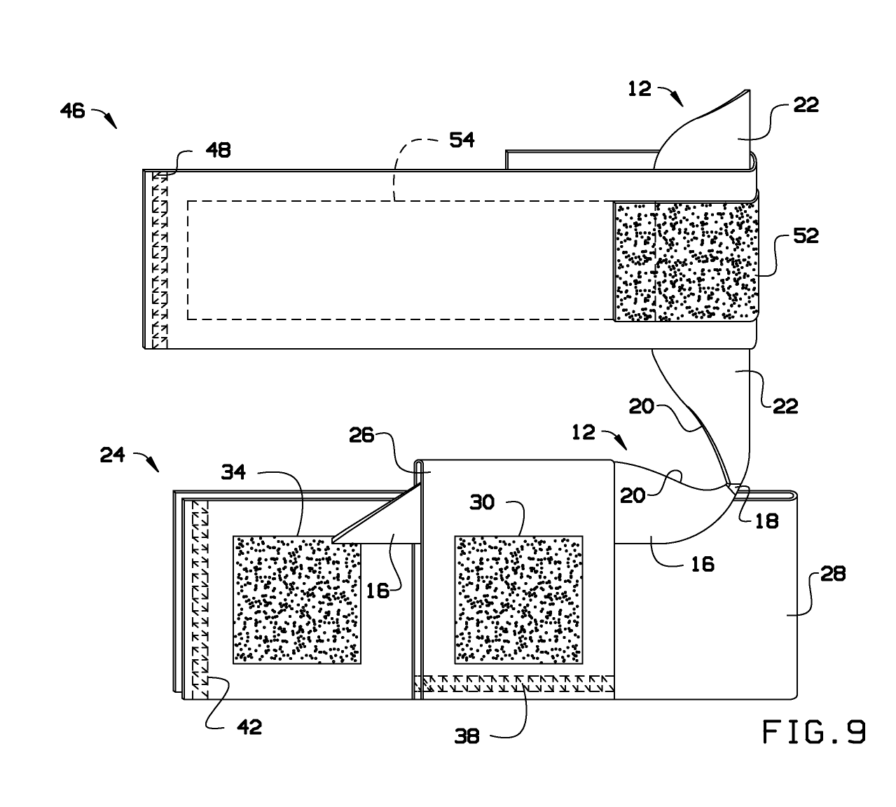

The humerus support section item 22 and the ulna and radius support section item 16 are the main sub-elements to create Olivia's Splint in item 12 for better understanding shown in FIG. 3 with FIG. 4 being an enhanced version of FIG. 3. Olivia's Splint item 12 was designed in mind to force the user's injured arm to bend at a roughly sixty to hundred degree angle holding the bones, and/or muscular ligaments in their rightful settings shown for a better understanding and function in FIG. 1 and FIG. 9. The ulna and radius support section item 16 and the humerus support section item 22 are constructed in a semi tubular fashion to encase the arm for recovery and stability. The ulna and radius support section item 16 and the humerus support section item 22 shown in FIG. 2, and for better understanding FIG. 3 or FIG. 4 will keep the injury from misalignment and keep it stable, to prevent the user from causing further damage to the injury. Olivia's Splint, in its entirety, is shown for a better understanding of the functionality and application in FIG. 1, FIG. 2, FIG. 3, FIG. 4 and FIG. 9.

The decompression hole item 14 for the user's olecranon is created via the ulna and radius support section item 16 and by the humerus support section item 22. The decompression hole item 14 shown in FIG. 2, FIG. 3 and for a better understanding FIG. 1. does not interfere with the users natural pulse rhythms and disallows the pinching of nerves, stiffness, swelling, and joint pain within the olecranon of the ulna region in/and around the bursa and tissues. The inside flex section for the joint item 18 in illustration FIG. 2 and FIG. 3 is an engaging section to allow bending of the joints. The outside flex section for the joint item 20 in the illustration FIG. 2, FIG. 3, and for better understanding FIG. 4 is another engaging section to allow bending of the joint.

Although the above description contains many specificities, these should not be construed as limiting the scope of the invention but merely to provide illustrations for some of the presently preferred embodiments of this invention. For example, the device need not necessarily be constructed from various specific material(s) but may be molded or constructed from conventional methods of orthopedic devices and shaped differently.

Alternatively, there can be variations made to Olivia's Splint item 12 that should not limit the scope or the functionality of the device. For example the length and symmetry can vary in shape and in size for the ulna and radius support section item 16 and humerus support section item 22.

Alternatively, the decompression hole item 14 for the user's olecranon may not exist in the entirety of the invention, but does not limit the scope or the functionality of Olivia's Splint item 12.

Alternatively, there can be variations made to Olivia's Splint item 12 and all of Olivia's Splint's item 12 existing sub-elements, the decompression hole item 14, the ulna and radius support section item 16, the inside flex section item 18, the outside flex section item 20, and the humerus support section item 22, that does not need to use conventional material(s) and fabric(s) used for medical devices that will not limit or inhibit the scope of the invention. For example various percentage(s) of the material or fabric can be constructed of polyester, polyvinyl chloride, polytetrafluroethylene, other polymer(s), neoprene, nylon, spandex and/or other fabric(s) for Olivia's Splint item 12.

### C. Arm and Chest Band

The dimensions of the arm and chest band item 46 are sixteen and one quarter inches long, by three inches wide. All measurements quoted herein may be adjusted up or down within a 20% or preferably 10% range. On the front and rear of the arm and chest band item 46 is a hook and loop system attached to the arm and chest band. The arm and chest band's item 46 function is to prevent movement of the thoracic cavity, or arms that correlate to the injury of the user. The arm and chest band item 46 also helps to stabilize and support the weight of the arms, shoulders, and thoracic cavity. The arm and chest band item 46 will also help prevent the misalignment of bones, and/or any strain of muscular ligaments and/or tendons. The arm and chest band item 46 acts as a cast and a brace. The arm and chest band item 46 acts as a guard against irritants to the skin. The arm and chest band item 46 acts as a positioning tool. The arm and chest band item 46 allows for anticipated expansion of the subject's physical anatomy. The arm and chest band item 46 is created in mind to provide a comfortable, non-hazardous material for infants, and a safe orthopedic brace for infants and/or children.

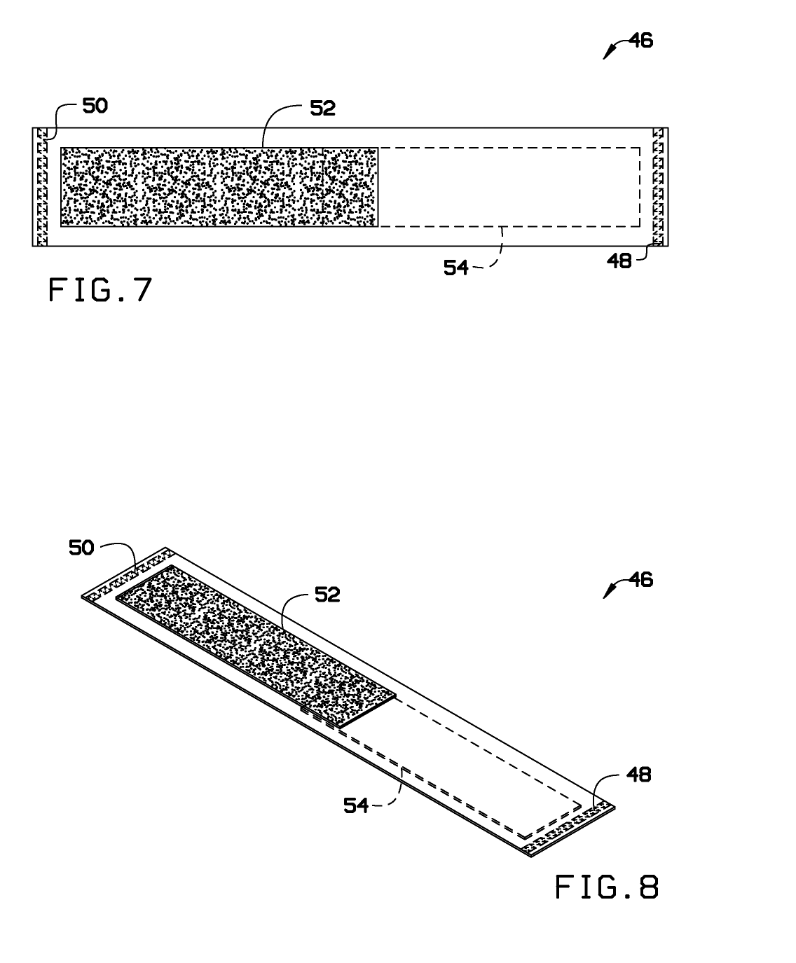

The arm and chest band item 46 shown in the illustration of FIG. 7 and for better understanding FIG. 8 is composed of a band that is made up of conventionally used orthopedic fabric(s) and material(s) and/or non-conventionally used orthopedic fabric(s) and material(s). The arm and chest band item 46 consists of an hook and loop system shown as item 52 and item 54, such as Velcro®.

The hook and loop system attached to the band item 46 on the front is item 52 shown in illustration FIG. 7 and for better understanding FIG. 8. The hook and loop system attached to the band item 46 on the rear is item 54 shown in the illustration of FIG. 7 and with better illustration FIG. 8. The hook and loop system is used to further stabilize and support Olivia's Splint item 46 shown in the illustration of FIG. 9 and for better understanding FIG. 1. The hook and loop system stabilizes and splints the humerus support section item 22 of Olivia's Splint item 12 shown in FIG. 1 and FIG. 9 the illustrations do not limit the scope of the invention. The arm and chest band item 46 has a right end seam item 48 and a left end seam item 50 illustrated in FIG. 7 and for better understanding FIG. 8.

Although the descriptions of the arm and chest band item 46 contain many specificities, these should not be construed as limiting the scope of the invention but merely to provide illustration of some of the presently preferred embodiments of this invention. For example the arm and chest band item 46 is made up of conventionally used orthopedic fabric(s) and material(s). The arm and chest band item 46 can be made of non-conventionally used orthopedic fabric(s) and material(s) that does not limit the structure, scope or functionality of the arm and chest band item 46.

Alternatively, there can be variations made to the arm and chest band item 46 that should not limit the scope of the structure or functionality of the sub-element(s). For example the sub-element can be longer in length. As well as the sub-element can be shorter or wider in width.

Alternatively, there can be variations made to the arm and chest band item 46 and all of the arm and chest band item 46 existing sub-elements, item 48, item 50, item 52, and item 54 that does not need to use conventional material(s) and fabric(s) used for orthopedic devices that will not limit or inhibit the scope of the invention. For example various percentage(s) of the material(s) or fabric(s) can be constructed of polyester, cotton, neoprene, nylon, spandex, silk, and/or other fabric(s) for the arm and chest band item 46.

### D. Tummy Wrap

The tummy wrap mid-section band's item 28 shown in illustration FIG. 5 dimensions are nine and three eighths inches long by three and one eighth inches wide. The tummy wrap wrist band item 26 shown in illustration FIG. 5 is attached to the tummy wrap mid-section band item 28 at a vertical angle and the tummy wrap wrist band's item 26 dimensions are seven and a half inches long by three inches wide. The tummy wrap item 24 shown in the illustration FIG. 5 and with a better understanding FIG. 6 is made up of conventionally used orthopedic fabric(s) and materials) and/or non-conventionally used orthopedic fabric(s) and material(s). The tummy wrap item 24 consists of the tummy wrap mid-section band item 28 and the tummy wrap wrist band item 26 in the illustration of FIG. 5 and with a better understanding FIG. 6 of which it is attached.

There is a hook and loop system on the tummy wrap mid-section band item 28 attached to the front end side item 34 and the rear end side item 36 of the tummy wrap item 24. The tummy wrap wrist band item 26 also has a hook and loop system attached to the front end side item 30 and rear end side item 32 of the tummy wrap item 24. The tummy wrap item 24 shown in illustration FIG. 5 and for better understanding in FIG. 1, FIG. 6, and FIG. 9 function is to support and stabilize the weight of the arm, shoulder, and thoracic cavity. The tummy wrap item 24 shown in illustration FIG. 5 and for better understanding in FIG. 1, FIG. 6, and FIG. 9 is also used to prevent movement of the wrist and joints that correlate with any injuries done to the infant's or child's muscular ligament(s), tendon(s), and misalignment of bone(s). The tummy wrap item 24 shown in illustration FIG. 5 and for better understanding in FIG. 1, FIG. 6, and FIG. 9 allows for growth to occur for the physical dimensions of human anatomy within the thoracic cavity and extremities. This band is created in mind to provide a comfortable, non-hazardous material for infants, and to provide a safe orthopedic brace for infants or children.

The tummy wrap wrist band item 26 contains a top end seam item 38 and a bottom end seam item 40 of the tummy wrap item 24 illustrated in FIG. 5 and with a better understanding FIG. 6. The tummy wrap mid-section band item 24 consists of left end seam item 42 and a right end seam item 44 of the tummy wrap item 24 illustrated in FIG. 5 and with a better understanding FIG. 6.

Although the descriptions of the tummy wrap item 24 illustrated in FIG. 5 and with a better understanding FIG. 6 contains specificities, these should not be construed as limiting the scope of the structure or functionality of the invention but merely, to providing illustration of some of the presently preferred embodiments of this invention. For example the tummy wrap wrist band item 26 on the tummy wrap item 24 can be longer or shorter in width or length.

Alternatively, there can be variations made to the tummy wrap item 24 and all of the tummy wrap's item 24 existing sub-elements item 26, item 28, item 30, item 32, item 34, item 36, item 38, item 40, item 42, item 44, and that do not need to use conventional and/or non-conventional material(s) and fabric(s) used for orthopedic devices that will not limit or inhibit the scope of the invention. For example various percent(s) of the material(s) or fabric(s) can be made out of polyester, cotton, neoprene, nylon, spandex, silk, and/or other fabric(s).

Alternatively, the tummy wrap item 24 can have varying hook and loop systems or non-existing hook and loop systems that does not limit the scope of the invention but perform the same task. For example the hook and loop system(s) can be replaced with button(s), snap(s), and other various fastener(s).

Alternatively, the tummy wrap wrist band item 26 attached to the tummy wrap mid-section item 28 as sub-elements of the tummy wrap item 24 may be smaller or larger in width or length. For example the tummy wrap wrist band item 26 can be smaller in length.

Alternatively, the tummy wrap mid-section band item 28 a sub-element of tummy wrap item 24 may be smaller or larger in width or length. For example the tummy wrap mid-section band item 28 can be smaller in length.

### E. Connections of Main Elements and Sub-Elements of Invention

The ulna and radius support section item 16 and the humerus support section item 22 are the main sub-elements of Olivia's Splint item 12 illustrated in FIG. 2, FIG. 3, and FIG. 4, and for better understanding FIG. 1 and FIG. 9. The decompression hole item 14, the inside flex section item 18 for the joint and the outside flex section item 20 for the joint are constructed through the ulna and radius support section item 16 and the humerus support section item 22 all of these sub-elements are it illustrated in FIG. 2 and FIG. 3. When Olivia's Splint item 12 is properly manufactured via conventional fabrication method(s) used for orthopedic braces it will be encased by the arm and chest band item 46 shown in FIG. 9 and for better understanding FIG. 1.

The arm and chest band item 46 illustrated in FIG. 7 and FIG. 8 and for better understanding FIG. 9 and FIG. 1 is composed of right end seam item 48, left end seam item 50, hook and loop system item 52 and item 54. The arm and chest band item 46 has a right end seam item 48 and a left end seam item 50 shown in the illustration of FIG. 7 and for better understanding FIG. 8. The hook and loop system item 52 is attached to the rear of the arm and chest band item 46 near the end as illustrated in FIG. 7 and for better understanding FIG. 8. The hook and loop system item 54 is attached to the front of the arm and chest band item 46 near the end as illustrated in FIG. 7 and for better understanding FIG. 8. The arm and chest band item 46 will encase around and over Olivia's Splint item 12 illustrated in FIG. 9 and for better understanding FIG. 1. The arm and chest band will then attach to the infant's or child's fabric item 56.

The tummy wrap item 24 consists of the tummy wrap wrist band item 26 and tummy wrap mid-section band item 28 illustrated in FIG. 5, FIG. 6 and for a better understanding FIG. 9 and FIG. 1. The tummy wrap wrist band item 26 is attached to the tummy wrap mid-section band item 28 as sub-elements of the tummy wrap item 24 that is illustrated in and FIG. 1, FIG. 5, FIG. 6 and FIG. 9. The tummy wrap wrist band item 26 has a hook and loop system item 32 attached to the bottom as illustrated in FIG. 5 and FIG. 6. The tummy wrap wrist band item 26 also has a hook and loop system item 30 attached to the top as illustrated in FIG. 5, FIG. 6 and FIG. 9. The tummy wrap wrist band has a top end seam item 38 illustrated in FIG. 5, FIG. 6, and FIG. 9 and a bottom end seam item 40 illustrated in FIG. 5 and FIG. 6.

The tummy wrap mid-section band item 28 has a left end seam item 42 illustrated in FIG. 5, FIG. 6, and FIG. 9. and a right end seam item 44 illustrated in FIG. 5 and FIG. 6.

The tummy wrap mid-section band item 28 has a hook and loop system item 34 attached to the left end as illustrated in FIG. 5, FIG. 6, and FIG. 9. The tummy wrap mid-section band item 28 has a hook and loop system item 36 attached to the right end as illustrated in FIG. 5 and FIG. 6. The tummy wrap wrist band item 26 a sub-element of the tummy wrap item 24 will encase around and over Olivia's Splint item 12 by attaching to itself through the hook and loop systems at the top item 30 and at the bottom item 32 all of which is illustrated in FIG. 9 and for better understanding FIG. 1.

The tummy wrap mid-section band item 28 a sub-element of the tummy wrap item 24 will fold around the user's abdomen illustrated in FIG. 9 and for better understanding FIG. 1. The tummy wrap mid-section band item 28 will then attach itself together by the hook and loop systems at the left item 34 and at the right item 36 illustrated in FIG. 9 and for better understanding FIG. 1

Olivia's Splint item 12, arm and chest band item 46, and the tummy wrap item 24 when fully assembled will create the infant and Child Upper Extremity and Thoracic Stability Brace illustrated in FIG. 9 and FIG. 1.

### F. Alternative Embodiments of Invention

Alternatively, the tummy wrap wrist band item 26 and the tummy wrap mid-section band item 28 illustrated in FIG. 5 and for better understanding FIG. 6 may vary in vertical and horizontal angles. This should not limit or inhibit the scope of the structure or functionality of the invention but to provide as some examples.

Alternatively, the tummy wrap wrist band item 26 and the tummy wrap mid-section item 28 illustrated in FIG. 5, FIG. 6, FIG. 9, and for better understanding FIG. 1, are sub-elements of the tummy wrap item 24 illustrated in FIG. 5 can have varying dimension(s) which include, but not limited to, length, width, depth, and/or mass. This should not limit or inhibit the scope of the structure or functionality of the invention bat to provide as some examples.

Alternatively, the tummy wrap wrist band item 26 and the tummy wrap mid-section band item 28 illustrated in FIG. 5 and for better understanding FIG. 6 may or may not contain fabric(s), seam(s), material(s) and/or technique(s) to measure room for anticipated growth. For example seams can be attached to the arm and chest band to show and measure growth and tension. This should not limit or inhibit the scope of the structure or functionality of the invention but to provide as some examples.

Alternatively, the arm and chest band item 46 shown in illustration FIG. 7, FIG. 8 and for better understanding FIG. 1 and FIG. 9 may be used for other extremities and various injuries. This should not limit or inhibit the scope of the structure or functionality of the invention but to provide as some examples.

Alternatively the arm and chest band item 46 illustrated in FIG. 7 and for better understanding FIG. 1 may or may not incorporate a hook and loop system attached as part of the users apparel. This should not limit or inhibit the scope of the functionality of the invention but to provide as some examples.

Alternatively the humerus support section item 22 and the ulna and radius support section item 16 may or may not incorporate additional material. For example a hook and loop system that allows additional stability and attachment sections. This should not limit or inhibit the scope of the functionality of the invention but to provide as some examples.

Alternatively, the arm and chest band item 46 illustrated in FIG. 7 may, and/or may not, cross wrap around the humerus and over the users chest and/or diagonally wrap up and over the users opposing shoulder of the injured extremity. This should not limit or inhibit the scope of the functionality of the invention but to provide as some examples.

Alternatively, the arm and chest band item 46 illustrated in FIG. 7, FIG. 8, FIG. 9 and for better understanding FIG. 1, can have varying dimension(s) which include, but not limited to, length, width, depth, and/or mass. This should not limit or inhibit the scope of the structure or functionality of the invention but to provide as some examples.

Alternatively, the arm and chest band item 46 illustrated in FIG. 7, and for better understanding FIG. 8, may or may not contain fabric(s), seam(s), material(s) and/or technique(s) to measure room for anticipated growth. For example seams can be attached to the arm and chest band to show and measure growth and tension. This should not limit or inhibit the scope of the structure or functionality of the invention but to provide as some examples.

Alternatively, the Infant and Child Upper Extremity and Thoracic Stability Brace FIG. 1 and FIG. 9 can be designed to conveniently locate other various arteries for measuring and/or monitoring purposes without interfering with natural pulse rhythms of the user. This should not limit or inhibit the scope of the functionality of the invention but to provide as some examples.

Alternatively, the Infant and Child Upper Extremity and Thoracic Stability Brace can be used for dystocia conditions and various injuries. This should not limit or inhibit the scope of the structure or functionality of the invention but to provide as some examples.

Alternatively, Olivia's Splint item 12, the tummy wrap item 24 and the arm and chest band item 46 in their entirety illustrated in FIG. 1 and FIG. 9 may be altered and/or added for enhancing the scope of the invention. For example Olivia's Splint item 12 can be made of various sub-element(s). Another example is the arm and chest band item 46 can have various sub-element(s). Another example is the tummy wrap item 24 can have various sub-element(s). This should not limit or inhibit the scope of the functionality of the invention but to provide as some examples.

Alternatively, the tummy wrap item 24 and the arm and chest band item 46, shown fully assembled as the Infant and Child Upper Extremity and Thoracic Stability Brace in illustration FIG. 1 and FIG. 9 can be made of non-orthopedic material(s) conventional or non-conventional to the manufacturing of orthopedic devices. For example, the infant and Child Upper Extremity and Thoracic Stability Brace illustrated in FIG. 9 can be made from velvet, cotton, polyester, neoprene, nylon and any other various material(s) and/or fabric(s) via conventional or non-conventional manufacturing method(s) of orthopedic device(s). This should not limit or inhibit the scope of the structure or functionality of the invention but to provide as some examples.

Alternatively, the tummy wrap item 24 and the arm and chest band item 46 shown fully assembled as the infant and child Upper Extremity and Thoracic Stability Brace in illustration FIG. 1 and FIG. 9 can have different conventional and/or non-conventional attaching methods to the device. For example, the hook and loop systems item 32, item 34, item 36, item 38, item 52 and item 54 can have various fastening methods made from any combination(s) of buttons, adhesives, tie straps, zippers, snaps, and/or other materials, fabrics, and or fasteners conventional or non-conventional to the manufacturing of medical devices. This should not limit or inhibit the scope of the structure or functionality of the invention but to provide as some examples.

Alternatively, the ulna and radius support section item 16 and the humerus support section item 22 illustrated in FIG. 2, and FIG. 3 may and/or may not have singular and/or multiple convexities and/or concavities within the dimensional range and/or domain of the product. This should not limit or inhibit the scope of the functionality of the invention but to provide as some examples.

Alternatively, the ulna and radius support section item 16 and the humerus support section item 22 illustrated in FIG. 2 may and/or may not be manipulated to a concave and/or convex shape. This should not limit or inhibit the scope of the functionality of the invention but to provide as some examples.

Alternatively, Olivia's Splint item 12 illustrated in FIG. 2, tummy wrap item 24 illustrated in FIG. 5 and arm and chest band item 46 illustrated in FIG. 7, in their entirety, illustrated in FIG. 1 and FIG. 9 may vary in fastening method(s), location(s) and or number(s). This should not limit or inhibit the scope of the functionality of the invention but to provide as some examples.

Alternatively, the decompression hole item 14 illustrated in FIG. 2 can be geometrically irregular or symmetrical in shape(s), size, dimension(s), width, or and/or length. This should not limit or inhibit the scope of the structure or functionality of the invention but to provide as some examples.

Alternatively, the ulna and radius support section item 16 and the humerus support section item 22 illustrated in FIG. 2 can be made with, but not limited to, ventilation opening(s), rib(s), ridge(s), and/or vent(s) made according to conventional and/or nonconventional method(s) of manufacturing medical device(s). This should not limit or inhibit the scope of the structure or functionality of the invention but to provide as some examples.

Alternatively, the ulna and radius support section item 16 and the humerus support section item 22 illustrated in FIG. 2 may be curved, indented, protruded, concaved, and/or may convex around the perimeter of all and/or part of Olivia's Splint item 12 illustrated in FIG. 2. This should not limit or inhibit the scope of the structure or functionality of the invention but to provide as some examples.

Alternatively, Olivia's Splint item 12 illustrated in FIG. 2, FIG. 3, FIG. 4, FIG. 9 and for better understanding FIG. 1 may not have material around, attached, and/or encased over the splint item 12 in FIG. 9 via various conventional and/or nonconventional manufacturing method(s) of medical devices. This should not limit or inhibit the scope of the structure or functionality of the invention but to provide as some examples.

Alternatively, Olivia's Splint item 12, the tummy wrap item 24, the arm and chest band item 46 and other various sub-elements of the Infant and Child Upper Extremity and Thoracic Stability Brace may or may not be implemented, according to the Practitioner's decision based on the user's needs. This should not limit or inhibit the scope of the functionality of the invention but to provide as some examples.

Alternatively, the inside flex section item 18 and the outside flex section item 20 of item 16 and item 22 illustrated in FIG. 2, FIG. 3 and for better understanding FIG. 4 can geometrically be irregular or symmetrical with and/or without various size(s) according to conventional or non-conventional method(s) of manufacturing medical devices. This should not limit or inhibit the scope of the structure or functionality of the invention but to provide as some examples.

Alternatively, the seams characterized as, but not limited to, item 38, item 40, item 42, and item 44 of the tummy wrap item 24 illustrated in FIG. 5 and item 48 and item 50 of the arm and chest band item 46 illustrated in FIG. 7, in their entirety illustrated in FIG. 1 and for better understanding FIG. 9, may have, but not limited to, various dimension(s), measurement(s), width, length, depth(s), mass and material(s). This should not limit or inhibit the scope of the structure or functionality of the invention but to provide as some examples.

Alternatively, the seams characterized as, but not limited to, item 38, item 40, item 42, item 44 of the tummy wrap item 24 illustrated in FIG. 5 and item 48 and item 50 of the arm and chest band item 46 illustrated in FIG. 7, in their entirety illustrated in FIG. 1 and FIG. 9 for better understanding can be geometrically irregular and/or symmetrical with or without various size(s) according to conventional and non-conventional method(s) of manufacturing medical devices. This should not limit or inhibit the scope of the structure or functionality of the invention but to provide as some examples.

Alternatively, the seams characterized as, but not limited to, item 38, item 40, item 42, and item 44 of the tummy wrap item 24 illustrated in FIG. 5 and item 48 and item 50 of the arm and chest band item 46 illustrated in FIG. 7, in their entirety illustrated in FIG. 1 and for better understanding FIG. 9, can be, but not limited to, made with ventilation opening(s), rib(s), ridge(s), and vent(s) made according to conventional and non-conventional method(s) of manufacturing medical devices. This should not limit or inhibit the scope of the structure or functionality of the invention but to provide as some examples.

Alternatively, Olivia's Splint item 12 illustrated in FIG. 2, FIG. 3, FIG. 4, FIG. 9 and for better understanding FIG. 1, may be constructed without the decompression hole item 14, illustrated in FIG. 2 and FIG. 3. This should not limit or inhibit the scope of the structure or functionality of the invention but to provide as an example.

Alternatively, Olivia's Splint item 12 illustrated in FIG. 2, FIG. 3, FIG. 4, FIG. 9 and for better understanding FIG. 1, may be constructed out of conventional or non-conventional material(s) and method(s) used for manufacturing medical devices. For example, it may be constructed from polymer(s), plastic(s), metal(s), or other various material(s). This should not limit or inhibit the scope of the structure or functionality of the invention but to provide as some examples.

Alternatively, the ulna and radius support section item 16 and the humerus support section item 22 are sub-elements of Olivia's Splint item 12 and are illustrated in FIG. 2, and FIG. 3 can have variations of dimension(s) which include, but not limited to, length, width, depth, and/or mass of the invention. This should not limit or inhibit the scope of the structure or functionality of the invention but to provide as some examples.

Alternatively, the inside flex section item 18 and the outside flex section item 20 of item 16 and item 22 illustrated in FIG. 2, FIG. 3 and for better understanding FIG. 4, can exist separately or as a sum of the parts. This should not limit or inhibit the scope of the structure or functionality of the invention but to provide as some examples.

Alternatively, the inside flex section item 18 and the outside flex section item 20 of item 16 and item 22 illustrated in FIG. 2, FIG. 3 and for better understanding FIG. 4 can vary in dimension(s) which include, but not limited to, length, width, depth, and/or mass. This should not limit or inhibit the scope of the structure or functionality of the invention but to provide as some examples.

Alternatively, the ulna and radius support section item 16 and the humerus support section item 22 illustrated in FIG. 2, FIG. 3, FIG. 4, FIG. 9 and for better understanding FIG. 1, are sub-elements of Olivia's Splint item 12 illustrated in FIG. 2 that can have varying dimension(s) which include, but not limited to, length, width, depth, and/or mass. This should not limit or inhibit the scope of the structure or functionality of the invention but to provide as some examples.

Alternatively, the hook and loop system item 30, item 32, item 34, and item 36 of the tummy wrap item 24 illustrated in FIG. 5 and item 52, and item 54 of the arm and chest band item 46 illustrated in FIG. 7, and for better understanding in their entirety illustrated in FIG. 9, may have various dimension(s), measurement(s), width, and length. Various depth(s) may also take place such as adding or subtracting material(s). This should not limit or inhibit the scope of the structure or functionality of the invention but to provide as some examples.

Alternatively, the hook and loop system item 30, item 32, item 34, and item 36 of the tummy wrap item 24 illustrated in FIG. 5 and item 52, and item 54 of the arm and chest band item 46 illustrated in FIG. 7 can geometrically be irregular or symmetrical with and/or without various size(s) according to conventional or non-conventional method(s) of manufacturing medical devices. This should not limit or inhibit the scope of the structure or functionality of the invention but to provide as some examples.

Alternatively, the hook and loop system item 30, item 32, item 34, and item 36 of the tummy wrap item 24 illustrated in FIG. 5 and item 52, and item 54 of the arm and chest band item 46 illustrated in FIG. 7 can be made with ventilation opening(s), rib(s), ridge(s), and vent(s) made according to conventional and non-conventional method(s) of manufacturing medical devices. This should not limit or inhibit the scope of the structure or functionality of the invention but to provide as some examples.

Alternatively, the hook and loop system item 30, item 32, item 34, and item 36 of the tummy wrap item 24 illustrated in FIG. 5 and item 52, and item 54 of the arm and chest band item 46 illustrated in FIG. 7 can have varying attachment system(s) to secure one end(s) to other(s). This should not limit or inhibit the scope of the structure or functionality of the invention but to provide as some examples.

### G. Operation of Preferred Embodiment

The manner of the orthopedic device of this invention is comfortable, easy, convenient, versatile, and is designed to conveniently locate the brachial artery for measuring and/or monitoring purposes without interfering with the natural pulse rhythms of the user. First the caretaker must have the user's wounded extremity or damaged area including the bone(s), and/or muscular ligament(s) in the thoracic cavity held in their rightful settings. Then the caretaker must take Olivia's Splint item 12 illustrated in FIG. 2 and align it so that the decompression hole item 14 is positioned over the olecranon of the ulna region in and around the bursa and tissues. The caretaker must then take Olivia's Splint item 12 and splint the injury with item 12. The user will splint the injury by taking Olivia's Splint item 12 illustrated in FIG. 2 and correctly align the wound and encase the extremity evenly in humerus support section item 22 and radius support section item 16. When correctly applied Olivia's Splint item 12 illustrated in FIG. 1 will be at relatively a sixty to hundred degree angle, or more preferably 70 to 90 degrees, for appropriate splinting of the damaged extremity.

The correct angle of the splint formation for Olivia's Splint item 12 is illustrated in FIG. 2 and for better understanding FIG. 4, FIG. 9 and FIG. 1. If an area in the thoracic cavity is injured the brace can be used without Olivia's Splint item 12. It is highly recommended that the brace is used with Olivia's Splint item 12 at all times to have full support and stability of the infant or child's injured area. The user must then take the arm and chest band item 46 illustrated in FIG. 1 and wrap the extremity fully to itself with the hook and loop system item 52 and the hook and loop system item 54. The arm and chest band item 46 illustrated in FIG. 1 will go across the chest of the user and attach to the user's fabric incorporating the remaining hook and loop system. Also the user must take the arm and chest band item 46 illustrated in FIG. 1 and wrap the extremity fully to itself with the hook and loop system item 52 and item 54 by going across the chest of the user to the shoulder. The caretaker must then take the tummy wrap mid-section band item 28 and place it underneath the user's back so that both hook and loop systems item 34 and item 36 attach evenly together. The caretaker must then place the user's ulna and radius region into the tummy wrap wrist band item 26. The caretaker must then use the hook and loop systems item 30 and item 32 and attach them together to securely fasten the user's extremity Olivia's Splint item 12, the tummy wrap item 24, and the arm and chest band item 46 show a fully assembled infant and Child Upper Extremity and Thoracic Stability Brace without a user in illustration FIG. 9, also for better understanding shown in FIG. 1, with a user.

Securing, splinting, and protecting the extremity and/or thoracic cavity with Olivia's Splint item 12, the tummy wrap item 24, and the arm and chest band item 46 using Olivia's Hybrid. Model allows for advantageous results conducive to recovery. The damaged area is allowed to recover faster because it no longer uses primitive healing techniques that have been used for centuries. An example of an old technique is immobilizing a user's extremity or thoracic cavity with gauze and/or a fastener(s) for dystocia conditions and other infant injuries. The method allowed for the user's bone(s) or damaged area to constantly misalign and consistently become agitated, preventing advantageous results conducive to recovery and unsustainable pain. Through the use of Olivia's Hybrid Model in Olivia's Splint item 12, the tummy wrap item 24, and the arm and chest band item 46 of the infant and Child Upper Extremity and Thoracic Stability Brace provides further stabilization of the injured area, as well as preventing unsustainable pain, negatively perceived inflictions while simultaneously incorporating the objectives of mechanical statics, positioning, pressure, and anticipated expansion of the subject's physical anatomy. The tummy wrap item 24 also prevents the infant or caretaker from untimely removing the arm and chest band item 46 and Olivia's Splint item 12 that may inhibit the functionality of the Infant/Child Upper Extremity And Thoracic Stability Brace shown in FIG. 1. The Infant/Child Upper Extremity And Thoracic Stability Brace allows for advantageous results conducive to recovery, significantly less unsustainable pain endured by the user, a design that provides convenient location(s) of the brachial artery for measuring and/or monitoring purposes without interfering with the natural pulse rhythms of the user, and is designed for comfortable, easy, convenient, and versatile use by the user and caretaker.

The Infant and Child Upper Extremity and Thoracic Stability Brace should be constructed using conventional methods similar to the methods used to manufacture orthopedic devices. Although the above descriptions contains many specifications, these should not be construed as limiting the scope of the invention but merely providing illustrations of some of the presently preferred embodiments of this invention. For example the fully assembled Infant/Child Upper Extremity And Thoracic Stability Brace shown in FIG. 9 and for better understanding in FIG. 1 may not use the tummy wrap item 24 if the Practitioner decides not to use it due to other formalities. Alternatively, Olivia's Splint may not be used in the fully assembled Infant/Child Upper Extremity And Thoracic Stability Brace if the Practitioner decides to not use it due to other formalities.

Applicant adds the following additional comments:
The invention is different from others because it is designed to target dystocia conditions in the thoracic and upper extremities regions of infants.
The Invention is different from others because it incorporates Olivia's splint.
   Olivia's Splint: a splint that incorporates mechanical statics, restriction of a body, and a positioning tool to provide increased protection, release of pressure from the Joint, room for anticipated growth and guard against negative inflictions,
The Invention is different from others because it mimics nursing and cradling positions.
The invention is different from others because it is designed to prevent irritants from reaching the skin.
The Invention is different from others because it allows growth to occur for the physical dimensions of human anatomy.
The Invention is different from others because it uses Olivia's Hybrid Model
   Olivia's Hybrid Model: An application that creates an advantageous symbiotic relationship that incorporates the principles of a cast, splint, and brace, while working against negatively perceived inflictions and simultaneously combining the objectives of mechanical statics, positioning, pressure, and anticipated expansion of the subject's physical anatomy for a specific purpose.
The invention is different from others because it completely encases the circumference of the ulna and radius region which limits accidental removal by the user and caretaker, keeps the user from deviating from the original position by using Olivia's Hybrid Model, and limits unsustainable pain and prevent further unnecessary negative inflictions.
The Invention is different from others because it completely encases the circumference of the humerus region ranging from, but not limited to, the axilla to the olecranon. It is then wrapped and bound into a fixed position which prevents negative inflictions to the user.
The Invention is different from others because the tummy wrap is fastened at the dorsal region of the user which prevents the user from undoing the hook and loop system.
The invention is different from others because it is designed to conveniently locate the brachial artery for measuring and/or monitoring purposes without interfering with the natural pulse rhythms of the user.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect.

### INDEX OF ELEMENTS

10: is the Exemplary User
12: is the Olivia's Splint
14: is the Olivia's Splint decompression hole
16: is the Olivia's Splint ulna radius support section
18: is the Olivia's Splint inside flex section
20: is the Olivia's Splint outside flex section
22: is the Olivia's Splint humerus support section
24: is the tummy wrap wrist band
26: is the tummy wrap wrist band
28: is the tummy wrap mid-section band
30: is the tummy wrap hook/loop system top of wrist band
32: is the tummy wrap hook/loop system bottom of wrist band
34: is the tummy wrap left hook/loop system attached to fabric
36: is the tummy wrap right hook/loop system attached to fabric
38: is the tummy wrap upper seam
40: is the tummy wrap lower seam
42: is the tummy wrap left seam
44: is the tummy wrap right seam
46: is the arm and chest band
48: is the atm and chest band right seam
50: is the atm and chest band left seam
52: is the arm and chest band hook/loop system attached to front of fabric
54: is the arm and chest band hook/loop system attached to rear of fabric
56: is the exemplary chest clothing article

1. A body recovery apparatus, comprising:
a splint comprising:
a humerus support section having a curved cross section;
an ulna radius support section having a curved cross section; and
a flex section flexibly connecting the humerus support section to the ulna radius support section,
wherein the humerus support section, ulna radius support section, and flex section are shaped and configured to define a decompression hole configured to align with a user's olecranon when the splint is applied to an arm of the user;
a chest band configured to enwrap the humerus support section of the splint and comprising a first chest band fastener configured to fasten to at least one of an apparel of the user and a second chest band fastener of the chest band; and
an abdomen wrap band comprising:
an abdomen band comprising a first abdomen band fastener configured to fasten to at least one of the apparel of the user and a second abdomen band fastener of the abdomen band; and
an ulna radius support section band connected to the abdomen band and comprising an ulna radius support section fastener configured to fasten to the ulna radius support section.

2. The body recovery apparatus as claimed in claim 1, wherein at least one of the fasteners comprises a hook-and-loop fastener.

3. The body recovery apparatus as claimed in claim 1, wherein the chest band comprises the second chest band fastener and the first chest band fastener is configured to fasten to the second chest band fastener.

4. The body recovery apparatus as claimed in claim 1, wherein the first chest band fastener is configured to fasten to the apparel of the user.

5. The body recovery apparatus as claimed in claim 1, wherein the abdomen band comprises the second abdomen band fastener and the first abdomen band fastener is configured to fasten to the second abdomen band fastener.

6. The body recovery apparatus as claimed in claim 1, wherein the first abdomen band fastener is configured to fasten to the apparel of the user.

7. A method of body recovery, comprising:
providing the body recovery apparatus as claimed in claim 1;
applying the splint to the arm of the user;
bending the splint at the flex section so that the humerus support section and ulna radius support section are angled relative to each other at an angle of between approximately 60 and 100 degrees;
fastening the chest band around the humerus support section;
fastening the first chest band fastener to at least one of the apparel of the user and the second chest band fastener of the chest band;
fastening the first abdomen band fastener to at least one of the apparel of the user and the second abdomen band fastener of the abdomen band; and
fastening the ulna radius support section fastener to the ulna radius support section.

8. The method as claimed in claim 7, wherein the user is an infant having a dystocia condition in at least one of a thoracic and upper extremity region.

9. The method as claimed in claim 7, further comprising completely encasing at least one of a circumference of the ulna region and a circumference of the humerus region.

10. The method as claimed in claim 7, wherein the four steps of fastening result in substantial immovability of the arm of the user.

11. The method as claimed in claim 7, wherein the step of fastening the first abdomen band fastener comprises fastening the first abdomen band fastener to the second abdomen band fastener at a back of the user.

12. The body healing recovery apparatus as claimed in claim 1, further comprising an absorbent material connected to at least one of the humerus support section and the ulna radius support section.

13. The method as claimed in claim 7, further comprising accessing the brachial artery while the body recovery apparatus is applied to the arm of the user.

14. The method as claimed in claim 7, further comprising adjusting the splint to achieve comfort of the user.

15. The method as claimed in claim 7, further comprising applying a scientific discipline of mechanical statics in executing the claimed steps.
